(19) United States
(12) Patent Application Publication
(10) Pub. No.: US 2003/0032638 A1
(43) Pub. Date: Feb. 13, 2003

(54) Delivery of benzodiazepines through an inhalation route

(76) Inventors: John J. Kim, San Jose, CA (US); Joshua D. Rabinowitz, Mountain View, CA (US); Alejandro C. Zaffaroni, Atherton, CA (US)

Correspondence Address: Richard R. Eckman, Morrison & Foerster LLP, 755 Page Mill Road, Palo Alto, CA, 94304-1018, US

(21) Appl. No.: 10/153,313

(22) Filed: May 21, 2002

Related U.S. Application Data
(60) Provisional application No. 60/294,203, filed on May 24, 2001.
(60) Provisional application No. 60/317,479, filed on Sep. 5, 2001.
(60) Provisional application No. 60/345,145, filed on Nov. 9, 2001.

(51) Int. Cl. A61K 31/5513; A61K 9/14; A61L 9/04

(52) U.S. Cl. 514/221; 424/46

(57) ABSTRACT

The present invention relates to the delivery of benzodiazepines through an inhalation route. Specifically, it relates to aerosols containing benzodiazepines that are used in inhalation therapy. In a composition aspect of the present invention, the aerosol comprises particles comprising at least 5 percent by weight of a benzodiazepine. In a method aspect of the present invention, a benzodiazepine is delivered to a mammal through an inhalation route. The method comprises: a) heating a composition, wherein the composition comprises at least 5 percent by weight of a benzodiazepine, to form a vapor; and, b) allowing the vapor to cool, thereby forming a condensation aerosol comprising particles, which is inhaled by the mammal. In a kit aspect of the present invention, a kit for delivering a benzodiazepine through an inhalation route to a mammal is provided which comprises: a) a composition comprising at least 5 percent by weight of a benzodiazepine; and, b) a device that forms a benzodiazepine containing aerosol from the composition, for inhalation by the mammal.

### CROSS-REFERENCE TO RELATED APPLICATIONS

This application claims priority to U.S. provisional application Ser. No. 60/294,203 entitled “Thermal Vapor Delivery of Drugs,” filed May 24, 2001, Rabinowitz and Zaffaroni, the entire disclosure of which is hereby incorporated by reference. This application further claims priority to U.S. provisional application Ser. No. 60/317,479 entitled “Aerosol Drug Delivery,” filed Sep. 5, 2001, Rabinowitz and Zaffaroni, the entire disclosure of which is hereby incorporated by reference. This application further claims priority to U.S. provisional application Ser. No. 60/345,145 entitled “Delivery of Clonazepam, Flunitrazepam and Flurazepam Through an Inhalation Route,” filed Nov. 9, 2001, Kim, the entire disclosure of which is hereby incorporated by reference.

### FIELD OF THE INVENTION

The present invention relates to the delivery of benzodiazepines through an inhalation route. Specifically, it relates to aerosols containing benzodiazepines that are used in inhalation therapy.

### BACKGROUND OF THE INVENTION

There are a number of compositions currently marketed for the treatment of seizure, panic disorders and insomnia. The compositions contain at least one active ingredient that provides for observed therapeutic effects. Among the active ingredients given in such compositions are clonazepam, flunitrazepam and flurazepam

It is desirable to provide a new route of administration for such compositions that rapidly produces peak plasma concentrations of the active ingredient. The provision of such a route is an object of the present invention.

### SUMMARY OF THE INVENTION

The present invention relates to the delivery of benzodiazepines through an inhalation route. Specifically, it relates to aerosols containing benzodiazepines that are used in inhalation therapy.

In a composition aspect of the present invention, the aerosol comprises particles comprising at least 5 percent by weight of a benzodiazepine. Preferably, the particles comprise at least 10 percent by weight of a benzodiazepine. More preferably, the particles comprise at least 20 percent, 30 percent, 40 percent, 50 percent, 60 percent, 70 percent, 80 percent, 90 percent, 95 percent, 97 percent, 99 percent, 99.5 percent or 99.97 percent by weight of a benzodiazepine.

Typically, the benzodiazepine is not one of the following benzodiazepines: adinazolam, chlordiazepoxide, clobenepam, lorazepam, loprazolam, midazolam, diazepam, alprazolam, estazolam, and triazolam.

Typically, the aerosol has a mass of at least 10 μg. Preferably, the aerosol has a mass of at least 100 μg. More preferably, the aerosol has a mass of at least 200 μg.

Typically, the aerosol particles comprise less than 10 percent by weight of benzodiazepine degradation products. Preferably, the particles comprise less than 5 percent by weight of benzodiazepine degradation products. More preferably, the particles comprise less than 2.5, 1, 0.5, 0.1 or 0.03 percent by weight of benzodiazepine degradation products.

Typically, the particles comprise less than 90 percent by weight of water. Preferably, the particles comprise less than 80 percent by weight of water. More preferably, the particles comprise less than 70 percent, 60 percent, 50 percent, 40 percent, 30 percent, 20 percent, 10 percent, or 5 percent by weight of water.

Typically, at least 50 percent by weight of the aerosol is amorphous in form, wherein crystalline forms make up less than 50 percent by weight of the total aerosol weight, regardless of the nature of individual particles. Preferably, at least 75 percent by weight of the aerosol is amorphous in form. More preferably, at least 90 percent by weight of the aerosol is amorphous in form.

Typically, the aerosol has an inhalable aerosol particle density greater than 10⁶ particles/mL. Preferably, the aerosol has an inhalable aerosol particle density greater than 10⁷ particles/mL. More preferably, the aerosol has an inhalable aerosol particle density greater than 10⁸ particles/mL.

Typically, the aerosol particles have a mass median aerodynamic diameter of less than 5 microns. Preferably, the particles have a mass median aerodynamic diameter of less than 3 microns. More preferably, the particles have a mass median aerodynamic diameter of less than 2 or 1 micron(s).

Typically, the geometric standard deviation around the mass median aerodynamic diameter of the aerosol particles is less than 3.0. Preferably, the geometric standard deviation is less than 2.5.

Typically, the aerosol is formed by heating a composition containing a benzodiazepine to form a vapor and subsequently allowing the vapor to condense into an aerosol.

In another composition aspect of the present invention, the aerosol comprises particles comprising at least 5 percent by weight of clonazepam, flunitrazepam or flurazepam. Preferably, the particles comprise at least 10 percent by weight of clonazepam, flunitrazepam or flurazepam. More preferably, the particles comprise at least 20 percent, 30 percent, 40 percent, 50 percent, 60 percent, 70 percent, 80 percent, 90 percent, 95 percent, 97 percent, 99 percent, 99.5 percent or 99.97 percent by weight of clonazepam, flunitrazepam or flurazepam.

Typically, the aerosol has a mass of at least 10 μg. Preferably, the aerosol has a mass of at least 100 μg. More preferably, the aerosol has a mass of at least 200 μg.

Typically, the aerosol particles comprise less than 10 percent by weight of clonazepam, flunitrazepam or flurazepam degradation products. Preferably, the particles comprise less than 5 percent by weight of clonazepam, flunitrazepam or flurazepam degradation products. More preferably, the particles comprise less than 2.5, 1, 0.5, 0.1 or 0.03 percent by weight of clonazepam, flunitrazepam or flurazepam degradation products.

Typically, the particles comprise less than 90 percent by weight of water. Preferably, the particles comprise less than 80 percent by weight of water. More preferably, the particles comprise less than 70 percent, 60 percent, 50 percent, 40 percent, 30 percent, 20 percent, 10 percent, or 5 percent by weight of water.

Typically, at least 50 percent by weight of the aerosol is amorphous in form, wherein crystalline forms make up less than 50 percent by weight of the total aerosol weight, regardless of the nature of individual particles. Preferably, at least 75 percent by weight of the aerosol is amorphous in form. More preferably, at least 90 percent by weight of the aerosol is amorphous in form.

Typically, where the aerosol comprises clonazepam or flunitrazepam, the aerosol has an inhalable aerosol drug mass density of between 0.3 mg/L and 10 mg/L. Preferably, the aerosol has an inhalable aerosol drug mass density of between 0.1 mg/L and 5 mg/L. More preferably, the aerosol has an inhalable aerosol drug mass density of between 0.2 mg/L and 2 mg/L.

Typically, where the aerosol comprises flurazepam, the aerosol has an inhalable aerosol drug mass density of between 0.03 mg/L and 50 mg/L. Preferably, the aerosol has an inhalable aerosol drug mass density of between 1 mg/L and 30 mg/L. More preferably, the aerosol has an inhalable aerosol drug mass density of between 4 mg/L and 20 mg/L.

Typically, the aerosol has an inhalable aerosol particle density greater than 10⁶ particles/mL. Preferably, the aerosol has an inhalable aerosol particle density greater than 10⁷ particles/mL. More preferably, the aerosol has an inhalable aerosol particle density greater than 10⁸ particles/mL.

Typically, the aerosol particles have a mass median aerodynamic diameter of less than 5 microns. Preferably, the particles have a mass median aerodynamic diameter of less than 3 microns. More preferably, the particles have a mass median aerodynamic diameter of less than 2 or 1 micron(s).

Typically, the geometric standard deviation around the mass median aerodynamic diameter of the aerosol particles is less than 3.0. Preferably, the geometric standard deviation is less than 2.5.

Typically, the aerosol is formed by heating a composition containing clonazepam, flunitrazepam or flurazepam to form a vapor and subsequently allowing the vapor to condense into an aerosol.

In a method aspect of the present invention, a benzodiazepine is delivered to a mammal through an inhalation route. The method comprises: a) heating a composition, wherein the composition comprises at least 5 percent by weight of a benzodiazepine; and, b) allowing the vapor to cool, thereby forming a condensation aerosol comprising particles, which is inhaled by the mammal. Preferably, the composition that is heated comprises at least 10 percent by weight of a benzodiazepine. More preferably, the composition comprises 20 percent, 30 percent, 40 percent, 50 percent, 60 percent, 70 percent, 80 percent, 90 percent, 95 percent, 97 percent, 99 percent, 99.5 percent, 99.9 percent or 99.97 percent by weight of a benzodiazepine.

Typically, the delivered aerosol particles comprise at least 5 percent by weight of a benzodiazepine. Preferably, the particles comprise at least 10 percent by weight of a benzodiazepine. More preferably, the particles comprise at least 20 percent, 30 percent, 40 percent, 50 percent, 60 percent, 70 percent, 80 percent, 90 percent, 95 percent, 97 percent, 99 percent, 99.5 percent, 99.9 percent or 99.97 percent by weight of a benzodiazepine.

Typically, the condensation aerosol has a mass of at least 10 μg. Preferably, the aerosol has a mass of at least 100 μg. More preferably, the aerosol has a mass of at least 200 μg.

Typically, the delivered aerosol particles comprise less than 10 percent by weight of benzodiazepine degradation products. Preferably, the particles comprise less than 5 percent by weight of benzodiazepine degradation products. More preferably, the particles comprise less than 2.5, 1, 0.5, 0.1 or 0.03 percent by weight of benzodiazepine degradation products.

Typically, the particles comprise less than 90 percent by weight of water. Preferably, the particles comprise less than 80 percent by weight of water. More preferably, the particles comprise less than 70 percent, 60 percent, 50 percent, 40 percent, 30 percent, 20 percent, 10 percent, or 5 percent by weight of water.

Typically, at least 50 percent by weight of the aerosol is amorphous in form, wherein crystalline forms make up less than 50 percent by weight of the total aerosol weight, regardless of the nature of individual particles. Preferably, at least 75 percent by weight of the aerosol is amorphous in form. More preferably, at least 90 percent by weight of the aerosol is amorphous in form.

Typically, the particles of the delivered condensation aerosol have a mass median aerodynamic diameter of less than 5 microns. Preferably, the particles have a mass median aerodynamic diameter of less than 3 microns. More preferably, the particles have a mass median aerodynamic diameter of less than 2 or 1 micron(s).

Typically, the geometric standard deviation around the mass median aerodynamic diameter of the aerosol particles is less than 3.0. Preferably, the geometric standard deviation is less than 2.5.

Typically, the delivered aerosol has an inhalable aerosol particle density greater than 10⁶ particles/mL. Preferably, the aerosol has an inhalable aerosol particle density greater than 10⁷ particles/mL. More preferably, the aerosol has an inhalable aerosol particle density greater than 10⁸ particles/mL.

Typically, the rate of inhalable aerosol particle formation of the delivered condensation aerosol is greater than 10⁸ particles per second. Preferably, the aerosol is formed at a rate greater than 10⁹ inhalable particles per second. More preferably, the aerosol is formed at a rate greater than 10¹⁰ inhalable particles per second.

Typically, the delivered aerosol is formed at a rate greater than 0.25 mg/second. Preferably, the aerosol is formed at a rate greater than 0.5 mg/second. More preferably, the aerosol is formed at a rate greater than 1 or 2 mg/second.

Typically, the delivered condensation aerosol results in a peak plasma concentration of the benzodiazepine in the mammal in less than 1 h. Preferably, the peak plasma concentration is reached in less than 0.5 h. More preferably, the peak plasma concentration is reached in less than 0.2, 0.1, 0.05, 0.02, 0.01, or 0.005 h (arterial measurement).

In another method aspect of the present invention, clonazepam, flunitrazepam or flurazepam is delivered to a mammal through an inhalation route. The method comprises: a) heating a composition, wherein the composition comprises at least 5 percent by weight of clonazepam, flunitrazepam or flurazepam; and, b) allowing the vapor to cool, thereby forming a condensation aerosol comprising particles, which is inhaled by the mammal. Preferably, the composition that is heated comprises at least 10 percent by weight of clonazepam, flunitrazepam or flurazepam. More preferably, the composition comprises 20 percent, 30 percent, 40 percent, 50 percent, 60 percent, 70 percent, 80 percent, 90 percent, 95 percent, 97 percent, 99 percent, 99.5 percent, 99.9 percent or 99.97 percent by weight of clonazepam, flunitrazepam or flurazepam.

Typically, the delivered aerosol particles comprise at least 5 percent by weight of clonazepam, flunitrazepam or flurazepam. Preferably, the particles comprise at least 10 percent by weight of clonazepam, flunitrazepam or flurazepam. More preferably, the particles comprise at least 20 percent, 30 percent, 40 percent, 50 percent, 60 percent, 70 percent, 80 percent, 90 percent, 95 percent, 97 percent, 99 percent, 99.5 percent, 99.9 percent or 99.97 percent by weight of clonazepam, flunitrazepam or flurazepam.

Typically, the condensation aerosol has a mass of at least 10 μg. Preferably, the aerosol has a mass of at least 100 μg. More preferably, the aerosol has a mass of at least 200 μg.

Typically, the delivered aerosol particles comprise less than 10 percent by weight of clonazepam, flunitrazepam or flurazepam degradation products. Preferably, the particles comprise less than 5 percent by weight of clonazepam, flunitrazepam or flurazepam degradation products. More preferably, the particles comprise less than 2.5, 1, 0.5, 0.1 or 0.03 percent by weight of clonazepam, flunitrazepam or flurazepam degradation products.

Typically, the particles comprise less than 90 percent by weight of water. Preferably, the particles comprise less than 80 percent by weight of water. More preferably, the particles comprise less than 70 percent, 60 percent, 50 percent, 40 percent, 30 percent, 20 percent, 10 percent, or 5 percent by weight of water.

Typically, at least 50 percent by weight of the aerosol is amorphous in form, wherein crystalline forms make up less than 50 percent by weight of the total aerosol weight, regardless of the nature of individual particles. Preferably, at least 75 percent by weight of the aerosol is amorphous in form. More preferably, at least 90 percent by weight of the aerosol is amorphous in form.

Typically, the particles of the delivered condensation aerosol have a mass median aerodynamic diameter of less than 5 microns. Preferably, the particles have a mass median aerodynamic diameter of less than 3 microns. More preferably, the particles have a mass median aerodynamic diameter of less than 2 or 1 micron(s).

Typically, the geometric standard deviation around the mass median aerodynamic diameter of the aerosol particles is less than 3.0. Preferably, the geometric standard deviation is less than 2.5.

Typically, where the aerosol comprises clonazepam or flunitrazepam, the delivered aerosol has an inhalable aerosol drug mass density of between 0.03 mg/L and 10 mg/L. Preferably, the aerosol has an inhalable aerosol drug mass density of between 0.1 mg/L and 5 mg/L. More preferably, the aerosol has an inhalable aerosol drug mass density of between 0.2 mg/L and 2 mg/L.

Typically, where the aerosol comprises flurazepam, the delivered aerosol has an inhalable aerosol drug mass density of between 0.03 mg/L and 50 mg/L. Preferably, the aerosol has an inhalable aerosol drug mass density of between 1 mg/L and 5 mg/L. More preferably, the aerosol has an inhalable aerosol drug mass density of between 4 mg/L and 20 mg/L.

Typically, the delivered aerosol has an inhalable aerosol particle density greater than 10⁶ particles/mL. Preferably, the aerosol has an inhalable aerosol particle density greater than 10⁷ particles/mL. More preferably, the aerosol has an inhalable aerosol particle density greater than 10⁸ particles/mL.

Typically, the rate of inhalable aerosol particle formation of the delivered condensation aerosol is greater than 10⁸ particles per second. Preferably, the aerosol is formed at a rate greater than 10⁹ inhalable particles per second. More preferably, the aerosol is formed at a rate greater than 10¹⁰ inhalable particles per second.

Typically, the delivered aerosol is formed at a rate greater than 0.25 mg/second. Preferably, the aerosol is formed at a rate greater than 0.5 mg/second. More preferably, the aerosol is formed at a rate greater than 1 or 2 mg/second.

Typically, where the condensation aerosol comprises clonazepam, between 0.05 mg and 5 mg of clonazepam are delivered to the mammal in a single inspiration. Preferably, between 0.1 mg and 3.5 mg of clonazepam are delivered to the mammal in a single inspiration. More preferably, between 0.15 mg and 2 mg of clonazepam are delivered to the mammal in a single inspiration.

Typically, where the condensation aerosol comprises flunitrazepam, between 0.05 mg and 5 mg of flunitrazepam are delivered to the mammal in a single inspiration. Preferably, between 0.1 mg and 3.5 mg of flunitrazepam are delivered to the mammal in a single inspiration. More preferably, between 0.15 mg and 2 mg of flunitrazepam are delivered to the mammal in a single inspiration.

Typically, where the condensation aerosol comprises flurazepam, between 1.5 mg and 50 mg of flurazepam are delivered to the mammal in a single inspiration. Preferably, between 1.5 mg and 40 mg of flurazepam are delivered to the mammal in a single inspiration. More preferably, between 1.5 mg and 30 mg of flurazepam are delivered to the mammal in a single inspiration.

Typically, the delivered condensation aerosol results in a peak plasma concentration of clonazepam, flunitrazepam or flurazepam in the mammal in less than 1 h. Preferably, the peak plasma concentration is reached in less than 0.5 h. More preferably, the peak plasma concentration is reached in less than 0.2, 0.1, 0.05, 0.02, 0.01, or 0.005 h (arterial measurement).

Typically, where the delivered condensation aerosol comprises flunitrazepam or flurazepam, it is used to treat insomnia.

Typically, where the delivered condensation aerosol comprises clonazepam, it is used to treat seizure or panic disorders.

In a kit aspect of the present invention, a kit for delivering a benzodiazepine through an inhalation route to a mammal is provided which comprises: a) a composition comprising at least 5 percent by weight of a benzodiazepine; and, b) a device that forms a benzodiazepine containing aerosol from the composition, for inhalation by the mammal. Preferably, the composition comprises at least 10 percent by weight of benzodiazepine. More preferably, the composition comprises at least 20 percent, 30 percent, 40 percent, 50 percent, 60 percent, 70 percent, 80 percent, 90 percent, 95 percent, 97 percent, 99 percent, 99.5 percent, 99.9 percent or 99.97 percent by weight of a benzodiazepine

Typically, the device contained in the kit comprises: a) an element for heating the benzodiazepine composition to form a vapor; b) an element allowing the vapor to cool to form an aerosol; and, c) an element permitting the mammal to inhale the aerosol.

In another kit aspect of the present invention, a kit for delivering clonazepam, flunitrazepam or flurazepam through an inhalation route to a mammal is provided which comprises: a) a composition comprising at least 5 percent by weight of clonazepam, flunitrazepam or flurazepam; and, b) a device that forms a clonazepam, flunitrazepam or flurazepam containing aerosol from the composition, for inhalation by the mammal. Preferably, the composition comprises at least 10 percent by weight of clonazepam, flunitrazepam or flurazepam. More preferably, the composition comprises at least 20 percent, 30 percent, 40 percent, 50 percent, 60 percent, 70 percent, 80 percent, 90 percent, 95 percent, 97 percent, 99 percent, 99.5 percent, 99.9 percent or 99.97 percent by weight of clonazepam, flunitrazepam or flurazepam.

Typically, the device contained in the kit comprises: a) an element for heating the clonazepam, flunitrazepam or flurazepam composition to form a vapor; b) an element allowing the vapor to cool to form an aerosol; and, c) an element permitting the mammal to inhale the aerosol.

### BRIEF DESCRIPTION OF THE FIGURE

FIG. 1 shows a device used to deliver benzodiazepine containing aerosols to a mammal through an inhalation route.

### DETAILED DESCRIPTION OF THE INVENTION

Definitions

“Aerodynamic diameter” of a given particle refers to the diameter of a spherical droplet with a density of 1 g/mL (the density of water) that has the *same settling velocity as the given particle.

“Aerosol” refers to a suspension of solid or liquid particles in a gas.

“Aerosol drug mass density” refers to the mass of benzodiazepine per unit volume of aerosol.

“Aerosol mass density” refers to the mass of particulate matter per unit volume of aerosol.

“Aerosol particle density” refers to the number of particles per unit volume of aero sol.

“Amorphous particle” refers to a particle that does not contain more than 50 percent by weight of a crystalline form. Preferably, the particle does not contain more than 25 percent by weight of a crystalline form. More preferably, the particle does not contain more than 10 percent by weight of a crystalline form.

“Benzodiazepine” degradation product refers to a compound resulting from a chemical modification of a benzodiazepine. The modification, for example, can be the result of a thermally or photochemically induced reaction. Such reactions include, without limitation, oxidation and hydrolysis.

“Clonazepam” refers to 5-(2-chlorophenyl)-1,3-dihydro-7-nitro-2H-1,4-benzodiazepin-2-one.

“Clonazepam degradation product” refers to a compound resulting from a chemical modification of clonazepam. The modification, for example, can be the result of a thermally or photochemically induced reaction. Such reactions include, without limitation, oxidation and hydrolysis.

“Condensation aerosol” refers to an aerosol formed by vaporization of a substance followed by condensation of the substance into an aerosol.

“Flunitrazepam” refers to 5-(2-fluorophenyl)-1,3-dihydro-1-methyl-7-nitro-1,4-benzodiazepin-2-one.

“Flunitrazepam degradation product” refers to a compound resulting from a chemical modification of flunitrazepam. The modification, for example, can be the result of a thermally or photochemically induced reaction. Such reactions include, without limitation, oxidation and hydrolysis.

“Flurazepam” refers to 7-chloro-1-(2-diethylaminoethyl)-5-(2-fluorophenyl)-1,3-dihydro-1,4-benzodiazepin-2-one.

“Flurazepam degradation product” refers to a compound resulting from a chemical modification of flurazepam. The modification, for example, can be the result of a thermally or photochemically induced reaction. Such reactions include, without limitation, oxidation and hydrolysis.

“Inhalable aerosol drug mass density” refers to the aerosol drug mass density produced by an inhalation device and delivered into a typical patient tidal volume.

“Inhalable aerosol mass density” refers to the aerosol mass density produced by an inhalation device and delivered into a typical patient tidal volume.

“Inhalable aerosol particle density” refers to the aerosol particle density of particles of size between 100 nm and 5 microns produced by an inhalation device and delivered into a typical patient tidal volume.

“Mass median aerodynamic diameter” or “MMAD” of an aerosol refers to the aerodynamic diameter for which half the particulate mass of the aerosol is contributed by particles with an aerodynamic diameter larger than the MMAD and half by particles with an aerodynamic diameter smaller than the MMAD.

“Rate of aerosol formation” refers to the mass of aerosolized particulate matter produced by an inhalation device per unit time.

“Rate of inhalable aerosol particle formation” refers to the number of particles of size between 100 nm and 5 microns produced by an inhalation device per unit time.

“Rate of drug aerosol formation” refers to the mass of aerosolized benzodiazepine produced by an inhalation device per unit time.

“Settling velocity” refers to the terminal velocity of an aerosol particle undergoing gravitational settling in air.

“Typical patient tidal volume” refers to 1 L for an adult patient and 15 mL/kg for a pediatric patient.

“Vapor” refers to a gas, and “vapor phase” refers to a gas phase. The term “thermal vapor” refers to a vapor phase, aerosol, or mixture of aerosol-vapor phases, formed preferably by heating.

Formation of Benzodiazepine Containing Aerosols

Any suitable method is used to form the aerosols of the present invention. A preferred method, however, involves heating a composition comprising a benzodiazepine to produce a vapor, followed by cooling of the vapor such that it condenses to provide a benzodiazepine comprising aerosol (condensation aerosol). The composition is heated in one of two forms: as pure active compound (e.g., pure clonazepam, flunitrazepam or flurazepam); or, as a mixture of active compound and a pharmaceutically acceptable excipient. Typically, the composition is heated on a solid support.

Pharmaceutically acceptable excipients are either volatile or nonvolatile. Volatile excipients, when heated, are concurrently volatilized, aerosolized and inhaled with the benzodiazepine. Classes of such excipients are known in the art and include, without limitation, gaseous, supercritical fluid, liquid and solid solvents. The following is a list of exemplary carriers within the classes: water; terpenes, such as menthol; alcohols, such as ethanol, propylene glycol, glycerol and other similar alcohols; dimethylformamide; dimethylacetamide; wax; supercritical carbon dioxide; dry ice; and mixtures thereof.

Solid supports on which the composition is heated are of a variety of shapes. Examples of such shapes include, without limitation, cylinders of less than 1.0 mm in diameter, boxes of less than 1.0 mm thickness and virtually any shape permeated by small (e.g., less than 1.0 mm-sized) pores. Preferably, solid supports provide a large surface to volume ratio (e.g., greater than 100 per meter) and a large surface to mass ratio (e.g., greater than 1 cm² per gram).

A solid support of one shape can also be transformed into another shape with different properties. For example, a flat sheet of 0.25 mm thickness has a surface to volume ratio of approximately 8,000 per meter. Rolling the sheet into a hollow cylinder of 1 cm diameter produces a support that retains the high surface to mass ratio of the original sheet but has a lower surface to volume ratio (about 400 per meter).

A number of different materials are used to construct the solid supports. Classes of such materials include, without limitation, metals, inorganic materials, carbonaceous materials and polymers. The following are examples of the material classes: aluminum, silver, gold, stainless steel, copper and tungsten; silica, glass, silicon and alumina; graphite, porous carbons, carbon yarns and carbon felts; polytetrafluoroethylene and polyethylene glycol. Combinations of materials and coated variants of materials are used as well.

Where aluminum is used as a solid support, aluminum foil is a suitable material. Examples of silica, alumina and silicon based materials include amphorous silica S-5631 (Sigma, St. Louis, Mo.), BCR171 (an alumina of defined surface area greater than 2 m²/g from Aldrich, St. Louis, Mo.) and a silicon wafer as used in the semiconductor industry. Carbon yarns and felts are available from American Kynol, Inc., New York, N.Y. Chromatography resins such as octadecycl silane chemically bonded to porous silica are exemplary coated variants of silica.

The heating of the benzodiazepine compositions is performed using any suitable method. Examples of methods by which heat can be generated include the following: passage of current through an electrical resistance element; absorption of electromagnetic radiation, such as microwave or laser light; and, exothermic chemical reactions, such as exothermic salvation, hydration of pyrophoric materials and oxidation of combustible materials.

Delivery of Benzodiazepine Containing Aerosols

Benzodiazepine containing aerosols of the present invention are delivered to a mammal using an inhalation device. Where the aerosol is a condensation aerosol, the device has at least three elements: an element for heating a benzodiazepine containing composition to form a vapor; an element allowing the vapor to cool, thereby providing a condensation aerosol; and, an element permitting the mammal to inhale the aerosol. Various suitable heating methods are described above. The element that allows cooling is, in it simplest form, an inert passageway linking the heating means to the inhalation means. The element permitting inhalation is an aerosol exit portal that forms a connection between the cooling element and the mammal's respiratory system.

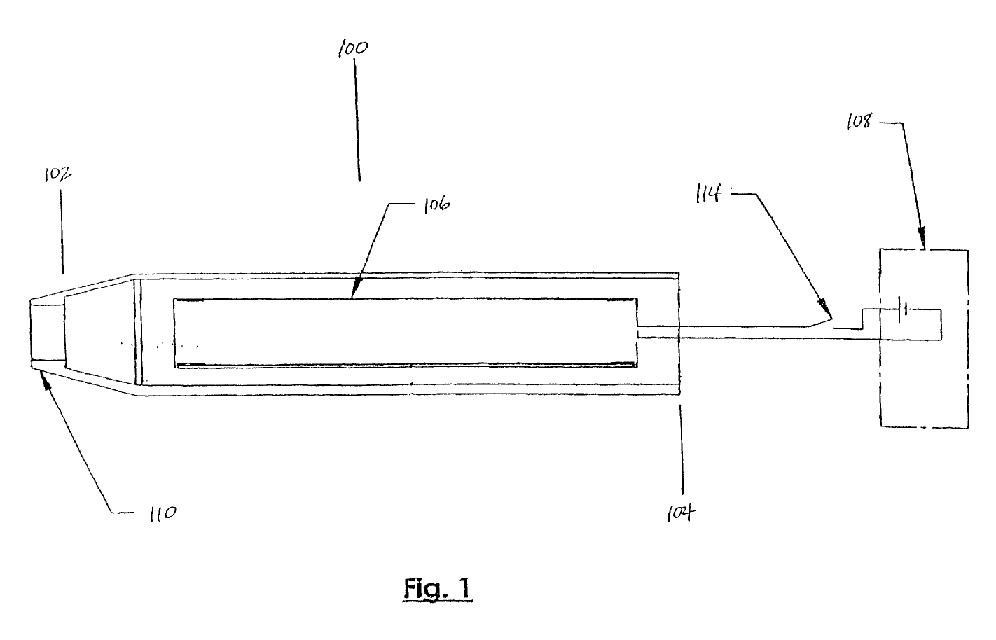

One device used to deliver a benzodiazepine containing aerosol is described in reference to FIG. 1. Delivery device 100 has a proximal end 102 and a distal end 104, a heating module 106, a power source 108, and a mouthpiece 110. A benzodiazepine composition is deposited on a surface 112 of heating module 106. Upon activation of a user activated switch 114, power source 108 initiates heating of heating module 106 (e.g, through ignition of combustible fuel or passage of current through a resistive heating element). The benzodiazepine composition volatilizes due to the heating of heating module 106 and condenses to form a condensation aerosol prior to reaching the mouthpiece 110 at the proximal end of the device 102. Air flow traveling from the device distal end 104 to the mouthpiece 110 carries the condensation aerosol to the mouthpiece 110, where it is inhaled by the mammal.

Devices, if desired, contain a variety of components to facilitate the delivery of benzodiazepine containing aerosols. For instance, the device may include any component known in the art to control the timing of drug aerosolization relative to inhalation (e.g., breath-actuation), to provide feedback to patients on the rate and/or volume of inhalation, to prevent excessive use (i.e., “lock-out” feature), to prevent use by unauthorized individuals, and/or to record dosing histories.

Dosage of Benzodiazepine Containing Aerosols

The dosage amount of a benzodiazepine in aerosol form is generally no greater than twice the standard dose of the drug given orally. For instance, for the treatment of seizure or panic disorders, clonazepam is given orally at strengths of 0.5 mg to 2 mg. Flunitrazepam and flurazepam are orally administered for the treatment of insomnia in doses of 0.5 mg to 2 mg and 15 mg or 30 mg respectively. As aerosols, 0.05 mg to 5 mg of clonazepam, 0.05 mg to 5 mg of flunitrazepam, and 1.5 mg to 50 mg of flurazepam are generally provided per inspiration for the same respective indications. A typical dosage of a benzodiazepine aerosol is either administered as a single inhalation or as a series of inhalations taken within an hour or less (dosage equals sum of inhaled amounts). Where the drug is administered as a series of inhalations, a different amount may be delivered in each inhalation.

One can determine the appropriate dose of a benzodiazepine containing aerosol to treat a particular condition using methods such as animal experiments and a dose-finding (Phase I/II) clinical trial. One animal experiment involves measuring plasma concentrations of drug in an animal after its exposure to the aerosol. Mammals such as dogs or primates are typically used in such studies, since their respiratory systems are similar to that of a human. Initial dose levels for testing in humans is generally less than or equal to the dose in the mammal model that resulted in plasma drug levels associated with a therapeutic effect in humans. Dose escalation in humans is then performed, until either an optimal therapeutic response is obtained or a dose-limiting toxicity is encountered.

Analysis of Benzodiazepine Containing Aerosols

Purity of a benzodiazepine containing aerosol is determined using a number of methods, examples of which are described in Sekine et al., Journal of Forensic Science 32:1271-1280 (1987) and Martin et al, Journal of Analytic Toxicology 13:158-162 (1989). One method involves forming the aerosol in a device through which a gas flow (e.g., air flow) is maintained, generally at a rate between 0.4 and 60 L/min. The gas flow carries the aerosol into one or more traps. After isolation from the trap, the aerosol is subjected to an analytical technique, such as gas or liquid chromatography, that permits a determination of composition purity.

A variety of different traps are used for aerosol collection. The following list contains examples of such traps: filters; glass wool; impingers; solvent traps, such as dry ice-cooled ethanol, methanol, acetone and dichloromethane traps at various pH values; syringes that sample the aerosol; empty, low-pressure (e.g., vacuum) containers into which the aerosol is drawn; and, empty containers that fully surround and enclose the aerosol generating device. Where a solid such as glass wool is used, it is typically extracted with a solvent such as ethanol. The solvent extract is subjected to analysis rather than the solid (i.e., glass wool) itself. Where a syringe or container is used, the container is similarly extracted with a solvent.

The gas or liquid chromatograph discussed above contains a detection system (i.e., detector). Such detection systems are well known in the art and include, for example, flame ionization, photon absorption and mass spectrometry detectors. An advantage of a mass spectrometry detector is that it can be used to determine the structure of benzodiazepine degradation products.

Particle size distribution of a benzodiazepine containing aerosol is determined using any suitable method in the art (e.g., cascade impaction). An Andersen Eight Stage Non-viable Cascade Inpactor (Andersen Instruments, Smyrna, Ga.) linked to a furnace tube by a mock throat (USP throat, Andersen Instruments, Smyrna, Ga.) is one system used for cascade impaction studies.

Inhalable aerosol mass density is determined, for example, by delivering a drug-containing aerosol into a confined chamber via an inhalation device and measuring the mass collected in the chamber. Typically, the aerosol is drawn into the chamber by having a pressure gradient between the device and the chamber, wherein the chamber is at lower pressure than the device. The volume of the chamber should approximate the tidal volume of an inhaling patient.

Inhalable aerosol drug mass density is determined, for example, by delivering a drug-containing aerosol into a confined chamber via an inhalation device and measuring the amount of active drug compound collected in the chamber. Typically, the aerosol is drawn into the chamber by having a pressure gradient between the device and the chamber, wherein the chamber is at lower pressure than the device. The volume of the chamber should approximate the tidal volume of an inhaling patient. The amount of active drug compound collected in the chamber is determined by extracting the chamber, conducting chromatographic analysis of the extract and comparing the results of the chromatographic analysis to those of a standard containing known amounts of drug.

Inhalable aerosol particle density is determined, for example, by delivering aerosol phase drug into a confined chamber via an inhalation device and measuring the number of particles of given size collected in the chamber. The number of particles of a given size may be directly measured based on the light-scattering properties of the particles. Alternatively, the number of particles of a given size may be determined by measuring the mass of particles within the given size range and calculating the number of particles based on the mass as follows: Total number of particles=Sum (from size range 1 to size range N) of number of particles in each size range. Number of particles in a given size range=Mass in the size range/Mass of a typical particle in the size range. Mass of a typical particle in a given size range=π*D³*φ/6, where D is a typical particle diameter in the size range (generally, the mean boundary MMADs defining the size range) in microns, φ is the particle density (in g/mL) and mass is given in units of picograms (g⁻¹²)

Rate of inhalable aerosol particle formation is determined, for example, by delivering aerosol phase drug into a confined chamber via an inhalation device. The delivery is for a set period of time (e.g., 3 s), and the number of particles of a given size collected in the chamber is determined as outlined above. The rate of particle formation is equal to the number of 100 nm to 5 micron particles collected divided by the duration of the collection time.

Rate of aerosol formation is determined, for example, by delivering aerosol phase drug into a confined chamber via an inhalation device. The delivery is for a set period of time (e.g., 3 s), and the mass of particulate matter collected is determined by weighing the confined chamber before and after the delivery of the particulate matter. The rate of aerosol formation is equal to the increase in mass in the chamber divided by the duration of the collection time. Alternatively, where a change in mass of the delivery device or component thereof can only occur through release of the aerosol phase particulate matter, the mass of particulate matter may be equated with the mass lost from the device or component during the delivery of the aerosol. In this case, the rate of aerosol formation is equal to the decrease in mass of the device or component during the delivery event divided by the duration of the delivery event.

Rate of drug aerosol formation is determined, for example, by delivering a benzodiazepine containing aerosol into a confined chamber via an inhalation device over a set period of time (e.g., 3 s). Where the aerosol is pure benzodiazepine, the amount of drug collected in the chamber is measured as described above. The rate of drug aerosol formation is equal to the amount of benzodiazepine collected in the chamber divided by the duration of the collection time. Where the benzodiazepine containing aerosol comprises a pharmaceutically acceptable excipient, multiplying the rate of aerosol formation by the percentage of benzodiazepine in the aerosol provides the rate of drug aerosol formation.

Utility of Benzodiazepine Containing Aerosols

Benzodiazepams are used for the treatment of a variety of indications, including seizure, muscle spasms, anxiety, nausea and panic attacks, insomnia, or sedation for medical or dental procedures.

The following examples are meant to illustrate, rather than limit, the present invention.

Clonazepam, flunitrazepam and flurazepam dihydrochloride were respectively obtained from Sigma (www.sigma-aldrich.com). Other benzodiazepines can be obtained from commercial sources or synthesized using standard methods in the art.

### EXAMPLE 1

### Volatilization of Clonazepam

A solution of 5 mg clonazepam in 120 μL dichloromethane was coated on a 3 cm×8 cm piece of aluminum foil. The dichloromethane was allowed to evaporate. The coated foil was wrapped around a 300 watt halogen tube (Feit Electric Company, Pico Rivera, Calif.), which was inserted into a glass tube sealed at one end with a rubber stopper. Running 60 V of alternating current (driven by line power controlled by a variac) through the bulb for 8 s afforded clonazepam thermal vapor (including clonazepam aerosol), which collected on the glass tube walls. Reverse-phase HPLC analysis with detection by absorption of 225 nm light showed the collected material to be at least 99.9% pure clonazepam.

### EXAMPLE 2

### Volatilization of Flunitrazepam

A solution of 5 mg flunitrazepam in 120 μL dichloromethane was coated on a 3 cm×8 cm piece of aluminum foil. The dichloromethane was allowed to evaporate. The coated foil was wrapped around a 300 watt halogen tube (Feit Electric Company, Pico Rivera, Calif.), which was inserted into a glass tube sealed at one end with a rubber stopper. Running 60 V of alternating current (driven by line power controlled by a variac) through the bulb for 7 s afforded flunitrazepam thermal vapor (including flunitrazepam aerosol), which collected on the glass tube walls. Reverse-phase HPLC analysis with detection by absorption of 225 nm light showed the collected material to be at least 99.9% pure flunitrazepam.

### EXAMPLE 3

### Volatilization of Flurazepam

A solution of 5 mg flurazepam in 120 μL dichloromethane was coated on a 3 cm×8 cm piece of aluminum foil. The dichloromethane was allowed to evaporate. The coated foil was wrapped around a 300 watt halogen tube (Feit Electric Company, Pico Rivera, Calif.), which was inserted into a glass tube sealed at one end with a rubber stopper. Running 60 V of alternating current (driven by line power controlled by a variac) through the bulb for 5 s afforded flurazepam thermal vapor (including flurazepam aerosol), which collected on the glass tube walls. Reverse-phase HPLC analysis with detection by absorption of 225 nm light showed the collected material to be at least 99.6% pure flurazepam. To obtain a higher purity aerosol, one can coat a lesser amount of drug, yielding a thinner film to heat. A linear decrease in film thickness is associated with a linear decrease in impurities.

### EXAMPLE 4

### Obtaining Flurazepam from Flurazepam Dihydrochloride

To 10 g flurazepam dihydrochloride was added 50 mL 1 N NaOH. The resulting solution was extracted 4 times with 50 mL dichloromethane. The organic layers were combined and washed with saturated aqueous NaCl solution. After collecting and drying the organic extract over Na₂SO₄, it was concentrated using a rotary evaporator with a bath temperature of 40° C. A yellow viscous liquid was obtained, which was approximately 99% pure flurazepam by HPLC.

### EXAMPLE 5

### Particle Size, Particle Density, and Rate of Inhalable Particle Formation of Flunitrazepam Aerosol

A solution of 1.7 mg flunitrazepam in 100 μL dichloromethane was spread out in a thin layer on the central portion of a 3.5 cm×7 cm sheet of aluminum foil. The dichloromethane was allowed to evaporate. The aluminum foil was wrapped around a 300 watt halogen tube, which was inserted into a T-shaped glass tube. Both of the openings of the tube were sealed with parafilm, which was punctured with fifteen needles for air flow. The third opening was connected to a 1 liter, 3-neck glass flask. The glass flask was further connected to a large piston capable of drawing 1.1 liters of air through the flask. Alternating current was run through the halogen bulb by application of 90 V using a variac connected to 110 V line power. Within 1 s, an aerosol appeared and was drawn into the 1 L flask by use of the piston, with collection of the aerosol terminated after 6 s. The aerosol was analyzed by connecting the 1 L flask to an eight-stage Andersen non-viable cascade impactor. Results are shown in table 1. MMAD of the collected aerosol was 1.7 microns with a geometric standard deviation of 2.4. Also shown in table 1 is the number of particles collected on the various stages of the cascade impactor, given by the mass collected on the stage divided by the mass of a typical particle trapped on that stage. The mass of a single particle of diameter D is given by the volume of the particle, πD³/6, multiplied by the density of the drug (taken to be 1 g/cm³). The inhalable aerosol particle density is the sum of the numbers of particles collected on impactor stages 3 to 8 divided by the collection volume of 1 L, giving an inhalable aerosol particle density of 1.7×10⁷ particles/mL. The rate of inhalable aerosol particle formation is the sum of the numbers of particles collected on impactor stages 3 through 8 divided by the formation time of 6 s, giving a rate of inhalable aerosol particle formation of 2.9×10⁹ particles/second.

TABLE 1
Determination of the characteristics of a flunitrazepam condensation
aerosol by cascade impaction using an Andersen 8-stage non-viable
cascade impactor run at 1 cubic foot per minute air flow.

| | | | Mass | |
|---|---|---|---|---|
| | Particle size | Average particle | collected | Number of |
| Stage | range (microns) | size (microns) | (mg) | particles |
| 0 | 9.0-10.0 | 9.5 | 0.004 | 2.0 × 10⁴ |
| 1 | 5.8-9.0 | 7.4 | 0.008 | 7.3 × 10⁴ |
| 2 | 4.7-5.8 | 5.25 | 0.008 | 3.4 × 10⁵ |
| 3 | 3.3-4.7 | 4.0 | 0.020 | 1.7 × 10⁶ |
| 4 | 2.1-3.3 | 2.7 | 0.056 | 1.1 × 10⁷ |
| 5 | 1.1-2.1 | 1.6 | 0.201 | 9.3 × 10⁷ |
| 6 | 0.7-1.1 | 0.9 | 0.163 | 2.2 × 10⁸ |
| 7 | 0.4-0.7 | 0.55 | 0.073 | 4.0 × 10⁸ |
| 8 | 0-0.4 | 0.2 | 0.090 | 1.6 × 10¹⁰ |

### EXAMPLE 6

### Drug Mass Density and Rate of Drug Aerosol Formation of Flunitrazepam Aerosol

A solution of 1.2 mg flunitrazepam in 100 μL dichloromethane was spread out in a thin layer on the central portion of a 3.5 cm×7 cm sheet of aluminum foil. The dichloromethane was allowed to evaporate. The aluminum foil was wrapped around a 300 watt halogen tube, which was inserted into a T-shaped glass tube. Both of the openings of the tube were sealed with parafilm, which was punctured with fifteen needles for air flow. The third opening was connected to a 1 liter, 3-neck glass flask. The glass flask was further connected to a large piston capable of drawing 1.1 liters of air through the flask. Alternating current was run through the halogen bulb by application of 90 V using a variac connected to 110 V line power. Within seconds, an aerosol appeared and was drawn into the 1 L flask by use of the piston, with formation of the aerosol terminated after 6 s. The aerosol was allowed to sediment onto the walls of the 1 L flask for approximately 30 minutes. The flask was then extracted with acetonitrile and the extract analyzed by HPLC with detection by light absorption at 225 nm. Comparison with standards containing known amounts of flunitrazepam revealed that 0.7 mg of >99% pure flunitrazepam had been collected in the flask, resulting in an aerosol drug mass density of 0.7 mg/L. The aluminum foil upon which the flunitrazepam had previously been coated was weighed following the experiment. Of the 1.2 mg originally coated on the aluminum, all of the material was found to have aerosolized in the 6 s time period, implying a rate of drug aerosol formation of 0.2 mg/s.

1. An aerosol for inhalation therapy, wherein the aerosol comprises particles comprising at least 10 percent by weight of a benzodiazepine, and wherein the benzodiazepine is not adinazolam, chlordiazepoxide, clobenepam, lorazepam, loprazolam, midazolam, diazepam, alprazolam, estazolam, or triazolam.

2. An aerosol for inhalation therapy, wherein the aerosol comprises particles comprising at least 10 percent by weight of clonazepam, flunitrazepam or flurazepam.

3. The aerosol according to claim 2, wherein the aerosol particles have a mass median aerodynamic diameter of less than 3 microns.

4. The aerosol according to claim 2, wherein the particles comprise less than 2.5 percent by weight of clonazepam, flunitrazepam or flurazepam degradation products.

5. The aerosol according to claim 3, wherein the aerosol comprises particles comprising at least 90 percent by weight of clonazepam, flunitrazepam or flurazepam.

6. The aerosol according to claim 5, wherein the aerosol particles have a mass median aerodynamic diameter less than 2 microns.

7. The aerosol according to claim 6, wherein the aerosol comprises particles comprising at least 97 percent by weight of clonazepam, flunitrazepam or flurazepam.

8. A method of delivering clonazepam, flunitrazepam or flurazepam to a mammal through an inhalation route, wherein the route comprises:
a) heating a composition, wherein the composition comprises at least 5 percent by weight of clonazepam, flunitrazepam or flurazepam, to form a vapor; and,
b) allowing the vapor to cool, thereby forming a condensation aerosol comprising particles,
which is inhaled by the mammal.

9. The method according to claim 8, wherein the particles comprise at least 10 percent by weight of clonazepam, flunitrazepam or flurazepam.

10. The method according to claim 8, wherein the aerosol particles have a mass median aerodynamic diameter of less than 3 microns.

11. The method according to claim 8, wherein the particles comprise less than 2.5 percent by weight of clonazepam, flunitrazepam or flurazepam degradation products.

12. The method according to claim 10, wherein the aerosol comprises particles comprising at least 90 percent by weight of clonazepam, flunitrazepam or flurazepam.

13. The method according to claim 12, wherein the aerosol particles have a mass median aerodynamic diameter less than 2 microns.

14. The method according to claim 13, wherein the aerosol comprises particles comprising at least 97 percent by weight of clonazepam, flunitrazepam or flurazepam.

15. A kit for delivering clonazepam, flunitrazepam or flurazepam through an inhalation route to a mammal, wherein the kit comprises:
a) a composition comprising at least 5 percent by weight of clonazepam, flunitrazepam or flurazepam; and,
b) a device that forms a clonazepam, flunitrazepam or flurazepam aerosol from the composition, for inhalation by the mammal
and wherein the device comprises:
a) an element for heating the clonazepam, flunitrazepam or flurazepam composition to form a vapor;
b) an element allowing the vapor to cool to form an aerosol; and,
c) an element permitting the mammal to inhale the aerosol.
